(19) United States
(12) Patent Application Publication
(10) Pub. No.: US 2005/0032687 A1
(43) Pub. Date: Feb. 10, 2005

(54) Method and composition for the modulation of angiogenesis

(76) Inventors: Joseph Loscalzo, Dover, MA (US); Jane A. Leopold, Chestnut Hill, MA (US)

Correspondence Address: Ronald I Eisenstein;Nixon Peabody, 100 Summer Street, Boston, MA, 02110, US

(21) Appl. No.: 10/493,289

(22) PCT Filed: Nov. 7, 2002
(86) PCT No.: PCT/US02/35821

Related U.S. Application Data
(60) Provisional application No. 60/344,974, filed on Nov. 7, 2001.

(51) Int. Cl. A61K 38/17; A61K 48/00

(52) U.S. Cl. 514/12; 514/44

(57) ABSTRACT

We have surprisingly discovered that the enzyme glucose-6-phosphate dehydrogenase (G6PD) regulates angiogenesis. As a result of this discovery, the present invention provides modulation of angiogenesis in tissues where that angiogenesis depends upon the activity of G6PD.

This invention was made in part with U.S. Government support under Contact Numbers P01 HL55993 and HL04399 awarded by the National Institutes of Health. The U.S. Government has certain rights in this application.

### FIELD OF THE INVENTION

The present invention provides for novel pharmaceutical compositions, and methods of use thereof for treatment of diseases or disorders involving angiogenesis.

### BACKGROUND OF THE INVENTION

Blood vessels are the means by which oxygen and nutrients are supplied to living tissues and waste products are removed from living tissue. Angiogenesis refers to the process by which new blood vessels are formed. (1; reviewed by Folkman, J., 2001, Semin. Oncol. 28 (6): 536-42; Ribatti, D., et al., 2000, Gen. Pharmacol. 35 (5): 227-31). Thus, where appropriate, angiogenesis is a critical biological process. It is essential in reproduction, development and wound repair. However, inappropriate angiogenesis can have severe negative consequences. For example, it is only after many solid tumors are vascularized as a result of angiogenesis that the tumors have a sufficient supply of oxygen and nutrients that permit it to grow rapidly and metastasize. Because maintaining the rate of angiogenesis in its proper equilibrium is so critical to a range of functions, it must be carefully regulated in order to maintain health. The angiogenesis process is believed to begin with the degradation of the basement membrane by proteases secreted from endothelial cells (EC) activated by mitogens such as vascular endothelial growth factor (VEGF) and basic fibroblast growth factor (bFGF). The cells migrate and proliferate, leading to the formation of solid endothelial cell sprouts into the stromal space, then, vascular loops are formed and capillary tubes develop with formation of tight junctions and deposition of new basement membrane.

In adults, the proliferation rate of endothelial cells is typically low compared to other cell types in the body. The turnover time of these cells can exceed one thousand days. Physiological exceptions in which angiogenesis results in rapid proliferation typically occurs under tight regulation, such as found in the female reproductive system and during wound healing.

The rate of angiogenesis involves a change in the local equilibrium between positive and negative regulators of the growth of microvessels. The therapeutic implications of angiogenic growth factors were first described by Folkman and colleagues over two decades ago (2). Abnormal angiogenesis occurs when there are increased or decreased stimuli for angiogenesis resulting in either excessive or insufficient blood vessel growth, respectively. For instance, conditions such as ulcers, strokes, and heart attacks may result from the absence of angiogenesis normally required for natural healing. In contrast, excessive blood vessel proliferation can result in tumor growth, tumor spread, blindness, psoriasis and rheumatoid arthritis.

Thus, there are instances where a greater degree of angiogenesis is desirable—increasing blood circulation, wound healing, and ulcer healing. For example, investigations have established the feasibility of using recombinant angiogenic growth factors, such as fibroblast growth factor (FGF) family (3, 4), endothelial cell growth factor (ECGF) (5), and more recently, vascular endothelial growth factor (VEGF) to expedite and/or augment collateral artery development in animal models of myocardial and hindlimb ischemia (5, 6).

Conversely, there are instances, where inhibition of angiogenesis is desirable. For example, many diseases are driven by persistent unregulated angiogenesis, also sometimes referred to as “neovascularization.” In arthritis, new capillary blood vessels invade the joint and destroy cartilage. In diabetes, new capillaries invade the vitreous, bleed, and cause blindness. Ocular neovascularization is the most common cause of blindness. Tumor growth and metastasis are angiogenesis-dependent A tumor must continuously stimulate the growth of new capillary blood vessels for the tumor itself to grow.

The current treatment of these diseases is inadequate. Agents which prevent continued angiogenesis, e.g, drugs (TNP470), monoclonal antibodies, antisense oligodeoxynucleotides and proteins (angiostatin (7), endostatin (8) and antiangiogenic ATIII (9)) are currently being tested. (10, 11, 12). Although preliminary results with the antiangiogenic proteins are promising, new antiangiogenic agents that show improvement in size, ease of production, stability and/or potency would be desirable.

### SUMMARY OF THE INVENTION

We have surprisingly discovered that the enzyme glucose-6-phosphate dehydrogenase (G6PD) regulates angiogenesis. As a result of this discovery, the present invention provides modulation of angiogenesis in tissues where that angiogenesis depends upon the activity of G6PD.

The present invention further provides compositions and methods for modulating angiogenesis in a tissue associated with a disease condition. A composition comprising an angiogenesis-modulating amount of a G6PD protein or an antagonist thereof is administered to tissue to be treated for a disease condition that responds to modulation of angiogenesis.

The composition providing the G6PD protein can contain purified protein, biologically active protein fragments, recombinantly produced G6PD protein or protein fragments or fusion proteins, or gene/nucleic acid expression vectors for expressing a G6PD protein.

The composition containing the G6PD antagonist can contain anti-G6PD antibodies, androsterone steroids, including dehydroepiandrosterone, and 16α-fluro-5α-androstan-17-one (FAO), as well as 6-aminonicotinamide and nicotinamide derivatives, RNAi, antisense oligoneuleotides, for example, antisense phosphorothioate oligodeoxynucleotides (5′-AGGUCACCCGAUGCACCCAUGAUGA-3′ (SEQ ID NO: 1)), including any sequence between 8-30 bases in any combination that results in inhibition of G6PD gene expression by hybrid arrest of translation or ribonuclease H digestion of the formed RNA-DNA heteroduplex. G6PD antagonists further include adenoviral-mediated overexpression of dominant negative forms of G6PD or G6PD cDNA with reduced activity. Proteins, muteins, peptides, mimetics and small molecule drugs that inhibit the angiogenic activity of G6PD can also be used.

The present invention can be used alone or in combination with other strategies to modulate angiogenesis.

Where the G6PD protein is inactivated or inhibited, the modulation is an inhibition of angiogenesis. Where the G6PD protein is active or activated, the modulation is a potentiation of angiogenesis. The tissue to be treated can be any tissue in which modulation of angiogenesis is desirable. For angiogenesis inhibition, it is useful to treat diseased tissue where deleterious neovascularization is occurring. Exemplary tissues include, solid tumors, metastases, tissues undergoing restenosis, and the like tissues. Inhibition of angiogenesis would also be beneficial in disease states characterized by pathological angiogenesis, such as tumor growth, diabetic retinopathy, vascular restenosis, primary pulmonary hypertension, hereditary hemorrhagic telangiectasis, post-operative adhesion formation, atherosclerosis and rheumatoid arthritis.

For potentiation, it is useful to treat patients with hypoxic tissues such as those following stroke, myocardial infarction or associated with chronic ulcers, tissues in patients with ischemic limbs in which there is abnormal, i.e., poor circulation, due to diabetic or other conditions. Patients with chronic wounds that do not heal, and therefore could benefit from the increase in vascular cell proliferation and neovascularization, can be treated as well. Potentiation of angiogenesis would also offer therapeutic benefit for ischemic vascular diseases, including coronary artery insufficiency and ischemic cardiomyopathy, peripheral arterial occlusive disease, cerebrovascular disease, ischemic bowel syndromes, impotence, and would healing.

The G6PD protein, peptide, and nucleic acid sequence encoding G6PD protein or peptide may be administered in conjunction with another angiogenesis stimulator.

The present invention also provides for methods to increase G6PD activity by means of administration of insulin or dietary manipulation.

The present invention also encompasses a pharmaceutical composition suitable for stimulating angiogenesis in a target mammalian tissue comprising a viral or non-viral gene transfer vector containg a nucleic acid, the nucleic acid having a nucleic acid segment encoding for a G6PD protein or peptide, and a pharmaceutically acceptable carrier.

Also envisioned is a pharmaceutical composition suitable for inhibiting angiogenesis in a target mammalian tissue and comprising a viral or non-viral gene transfer vector containing, for example, a nucleic acid having a segment encoding for an antisense oligonucleotide to G6PD mRNA and a pharmaceutically acceptable carrier.

Other aspects of the invention are disclosed infra.

### BRIEF DESCRIPTION OF THE DRAWINGS

FIGS. 1A and 1B show G6PD and endothelial cell proliferation. BAEC were treated with DHEA to decrease G6PD activity and stimulated with VEGF (100 ng/ml) for 8 hr (1A). Cell proliferation is measured by [³H]-Thymidine incorporation. G6PD expression was inhibited by trans(in)fection with an antisense oligonucleotide to G6PD mRNA (AS) or a scrambled control (SS) and proliferation was determined by [³H]-Thymidine incorporation (1B). Results are expressed as counts per minute (cpm) and data presented as mean±SEM. *p<0.01 vs.−VEGF.**p<0.01 vs.−DHEA or −SS.

FIGS. 2A and B show overexpression of G6PD using adenoviral gene transfer. G6PD expression is significantly increased following trans(in)fection with pAD-G6PD (2.5×MOI.) (2A). This correlates with a five-fold increase in G6PD activity (n=(2B). G6PD activity is expressed as units/6 min/mg protein and data are presented as mean±SEM. *p<0.01 vs. 0×MOI. MOI multiplicity of infection.

FIGS. 3A-D show G6PD and endothelial cell migration. BAEC were transfected with AS (3B) or the scrambled control, SS (3A), and observed to migrate from right to left across a vertical groove in the tissue culture plate. These representative frames demonstrate that VEGF increases BAEC migration in SS (3C), but not AS (3D), transfected cells, suggesting that G6PD activity is important for cell migration.

FIG. 4 shows G6PD and cell migration. Fluorescently labeled, BAEC were stimulated with VEGF (100ng/ml) for 18 hr and cell migration was assessed in a modified Boyden Chamber. In BAEC with normal G6PD activity, VEGF significantly increased cell migration as determined by increased fluorescence (13.3±2.6 vs. 55.3±6.6 units, p<0.01 vs.−VEGF, **p<0.01 vs.−DHEA.

FIGS. 5A and B show G6PD and Tube Formation. BAEC were transfected with an antisense oligodecocynucleotide to G6PD and mRNA (AS) or a scrambled control (SS) and plated on Matrigel. Cells were then stimulated with VEGF and tube formation was assessed visually (5A) and quantified using area analysis with background substration (5B). Results of area analysis are the average of 10 high-powered fields.

FIGS. 6A and B show G6PD overexpression and tube formation. G6PD expression was increased via adenoviral-mediated gene transfer. BAEC were then placed on Matrigel and stimulated with VEGF (100 nm/ml).

FIG. 7 shows the effect of VEGF on reactive oxygen species (ROS) production in BAEC.

FIG. 8 shows the effects of G6PD and NO on cell migration.

FIGS. 9A and B show a Matrigel plug from a WT mouse implanted 14 days at 20×(9A) and 4×(9B) magnification stained with H&E. M=Matrigel, Mus=muscle layer, A=subcutaneous adipose tissue. Arrow shows cells that have migrated into the Matrigel.

FIGS. 10A-D show G6PD and angiogenesis in vivo. H&E stain, 10×; WT=C3H wild-type; HEMI=G6PD X^{b}Y.

FIGS. 11A and B show that cells migrating into the Matrigel plugs stain positive for vWF antigen, demonstrating that they are endothelial cells.

FIGS. 12A-C show that cells migrating into the Matrigel plus stain positive for eNOS and Nox1 antigens (arrows). 20× magnification.

FIGS. 13A-D show that G6PD gene transfer rescues G6PD deficient phenotype (HEMI mouse) and promotes migration into Matrigel. No addition (13A); VEGF (13B); AdG6PD, adenovirus encoding for murine G6PD (13C); AdG6PD and VEGF (13D). H&E stain. Panels A and C are 4× and panels B and D are 10× magnification.

### DETAILED DESCRIPTION OF THE INVENTION

Angiogenesis, the formation of new blood vessels in response to tissue ischemia, is dependent upon a coordinated sequence of events involving vascular endothelial cell migration, proliferation and tube formation. Vascular redox state, reactive oxygen species (ROS) formation, and nitric oxide (NO) have been shown to modulate growth factor-mediated endothelial cell migration and proliferation. G6PD, the first and rate-limited enzyme in the pentose phosphate pathway that produces ribose moieties for nucleic acid synthesis, is the principal cellular source of NADPH. NADPH, in turn serves as a cofactor for enzymes that regulate the cellular flux of ROS and endothelial nitric oxide synthase (eNOS) activity. Therefore, in that G6PD is involved in the production of ROS and NO and thereby mediates endothelial cell proliferation and migration to effect new vessel formation, it is a candidate to target to modulate angiogenesis.

This discovery is important because of the role that angiogenesis plays in a variety of disease processes. On the other hand, where tissues associated with a disease condition require angiogenesis for tissue growth, it is desirable to inhibit angiogenesis and thereby inhibit the diseased tissue growth. Where injured tissue requires angiogenesis for tissue growth and healing, it is desirable to potentiate or promote angiogenesis and thereby promote tissue healing and growth.

Where the growth of new blood vessels is the cause of, or contributes to, the pathology associated with a disease tissue, inhibition of angiogenesis will reduce the deleterious effects of the disease. By inhibiting angiogenesis, one can intervene in the disease, ameliorate the symptoms, and in some cases cure the disease.

Examples of tissue associated with disease and neovascularization that will benefit from inhibitory modulation of angiogenesis include cancer, rheumatoid arthritis, ocular diseases such as diabetic retinopathy, inflammatory diseases, restenosis, and the like. Where the growth of new blood vessels is required to support growth of a deleterious tissue, inhibition of angiogenesis reduces the blood supply to the tissue and thereby contributes to reduction in tissue mass based on blood supply requirements. Particularly preferred examples include growth of tumors where neovascularization is a continual requirement in order that the tumor grow beyond a few millimeters in thickness, and for the establishment of solid tumor metastases.

Where the growth of new blood vessels contributes to healing of tissue, potentiation of angiogenesis assists in healing. Examples include treatment of patients with ischemic limbs in which there is abnormal, i.e. poor circulation as a result of diabetes or other conditions. Also contemplated are patients with chronic wounds which do not heal and therefore could benefit from the increase in vascular cell proliferation and neovascularization.

A G6PD protein for use in the present invention can vary depending upon the intended use. The terms “G6PD protein” or “G6PD” are used to refer collectively to the various forms of the enzyme, either in active or inactive forms.

An “active G6PD protein” refers to any of a variety of forms of the G6PD protein, including peptides, which potentiate, stimulate, activate, induce or increase angiogenesis. Assays to measure potentiation of angiogenesis are described herein, and are not to be construed as limiting. A protein is considered active if the level of angiogenesis is at least 10% greater, preferably 25% greater, and more preferably 50% greater than a control level where no G6PD is added to the assay system.

An “inactive G6PD protein” or “G6PD antagonist” refers to any of a variety of forms of G6PD protein or other compound which inhibit, reduce, impede, or restrict angiogenesis or inhibit expression or activity of G6PD. Assays to measure inhibition of angiogenesis are described herein, and are not to be construed as limiting. A protein or compound is considered inactive or an antagonist if the level of angiogenesis is at least 10% lower, preferably 25% lower, and more preferably 50% lower than a control level where no exogenous G6PD is added to the assay system.

A G6PD protein useful in the present invention can be produced in any of a variety of methods including isolation from natural sources including tissue, production by recombinant DNA expression and purification, and the like. G6PD protein can also be provided “in situ” by introduction of a gene therapy system to the tissue of interest which then expresses the protein in the tissue.

A gene encoding a G6PD protein can be prepared by a variety of methods known in the art. For example, the gene can readily be cloned using cDNA cloning methods from any tissue expressing the protein. The human cDNA accession number is M21248 and the rat cDNA accession number is NM_{—}017006. Protein accession numbers are NP_{—}000393 and NP_{—}058702 for human and rat respectively.

The invention provides nucleotide sequences of particular use in the present invention. These define nucleic acid sequences which encode for G6PD protein useful in the invention, and various DNA segments, recombinant DNA (rDNA) molecules and vectors constructed for expression of G6PD protein. DNA molecules (segments) of this invention therefore can comprise sequences which encode whole structural genes, fragments of structural genes, and transcription units.

A preferred DNA segment is a nucleotide sequence which encodes a G6PD protein as defined herein, or biologically active fragment thereof. By biologically active, it is meant that the expressed protein will have at least some of the biological activity of the intact protein found in a cell.

A preferred DNA segment codes for an amino acid residue sequence substantially the same as, and preferably consisting essentially of, an amino acid residue sequence or portions thereof corresponding to a G6PD protein described herein.

A nucleic acid is any polynucleotide or nucleic acid fragment, whether it be a polyribonucleotide of polydeoxyribonucleotide, i.e., RNA or DNA, or analogs thereof.

DNA segments are produced by a number of means including chemical synthesis methods and recombinant approaches, preferably by cloning or by polymerase chain reaction (PCR).

The G6PD gene of this invention can be cloned from a suitable source of genomic DNA or messenger RNA (mRNA) by a variety of biochemical methods. Cloning these genes can be conducted according to the general methods known in the art. Sources of nucleic acids for cloning a G6PD gene suitable for use in the methods of this invention can include genomic DNA or messenger RNA (mRNA) in the form of a cDNA library, from a tissue believed to express these proteins.

A preferred cloning method involves the preparation of a cDNA library using standard methods, and isolating the G6PD-encoding or nucleotide sequence by PCR amplification using paired oligonucleotide primers based on nucleotide sequences described herein. Alternatively, the desired cDNA clones can be identified and isolated from a cDNA or genomic library by conventional nucleic acid hybridization methods using a hybridization probe based on the nucleic acid sequences described herein. Other methods of isolating and cloning suitable G6PD-encoding nucleic acids are readily apparent to one skilled in the art.

The invention also includes a recombinant DNA molecule (rDNA)containing a DNA segment encoding a G6PD as described herein. An expressible rDNA can be produced by operatively (in frame, expressibly) linking a vector to a G6PD encoding DNA segment of the present invention.

The choice of vector to which a DNA segment of the present invention is operatively linked depends directly, as is well known in the art, on the functional properties desired, e.g., protein expression, and the host cell to be transformed. Both prokaryotic and eukaryotic expression vectors are familiar to one of ordinary skill in the art of vector construction, and are described by Sambrook et al., Molecular Cloning: A Laboratory Manual Cold Spring Harbor Laboratory (2001).

Expression vectors compatible with eukaryotic cells, preferably those compatible with vertebrate cells, can be used to form the recombinant DNA molecules of the present invention. Eukaryotic cell expression vectors are well known in the art and are available from several commercial sources. Typically, such vectors are provided containing convenient restriction sites for insertion of the desired DNA segment

Additionally, the angiogenesis modulator can also be delivered using gene therapy. The gene transfer methods for gene therapy fall into three broad categories: (1) physical (e.g., electroporation, direct gene transfer and particle bombardment), (2) chemical (e.g. lipid-based carriers and other non-viral vectors) and (3) biological (e.g. virus derived vectors). For example, non-viral vectors such as liposomes coated with DNA may be directly injected intravenously into the patient It is believed that the liposome/DNA complexes are concentrated in the liver where they deliver the DNA to macrophages and Kupffer cells.

Gene therapy methodologies can also be described by delivery site. Fundamental ways to deliver genes include ex vivo gene transfer, in vivo gene transfer, and in vitro gene transfer. In ex vivo gene transfer, cells are taken from the patient and grown in cell culture. The DNA is transfected into the cells, the transfected cells are expanded in number and then reimplanted in the patient. In in vitro gene transfer, the transformed cells are cells growing in culture, such as tissue culture cells, and not particular cells from a particular patient. These “laboratory cells” are transfected, the transfected cells are selected and expanded for either implantation into a patient or for other uses. In vivo gene transfer involves introducing the DNA into the cells of the patient when the cells are within the patient. All three of the broad based categories described above may be used to achieve gene transfer in vivo, ex vivo, and in vitro.

Mechanical (i.e. physical) methods of DNA delivery can be achieved by direct injection of DNA, such as microinjection of DNA into germ or somatic cells, pneumatically delivered DNA-coated particles, such as the gold particles used in a “gene gun,” and inorganic chemical approaches such as calcium phosphate transfection. It has been found that physical injection of plasmid DNA into muscle cells yields a high percentage of cells which are transfected and have a sustained expression of marker genes. The plasmid DNA may or may not integrate into the genome of the cells. Non-integration of the transfected DNA would allow the transfection and expression of gene product proteins in terminally differentiated, non-proliferative tissues for a prolonged period of time without fear of mutational insertions, deletions, or alterations in the cellular or mitochondrial genome. Long-term, but not necessarily permanent, transfer of therapeutic genes into specific cells may provide treatments for genetic diseases or for prophylactic use. The DNA could be reinjected periodically to maintain the gene product level without mutations occurring in the genomes of the recipient cells. Non-integration of exogenous DNAs may allow for the presence of several different exogenous DNA constructs within one cell with all of the constructs expressing various gene products.

Particle-mediated gene transfer may also be employed for injecting DNA into cells, tissues and organs. With a particle bombardment device, or “gene gun,” a motive force is generated to accelerate DNA-coated high density particles (such as gold or tungsten) to a high velocity that allows penetration of the target organs, tissues or cells. Electroporation for gene transfer uses an electrical current to make cells or tissues susceptible to electroporation-mediated gene transfer. A brief electric impulse with a given field strength is used to increase the permeability of a membrane in such a way that DNA molecules can penetrate into the cells. The techniques of particle-mediated gene transfer and electroporation are well known to those of ordinary skill in the art.

Chemical methods of gene therapy involve carrier mediated gene transfer through the use of fusogenic lipid vesicles such as liposomes or other vesicles for membrane fusion. A carrier harboring a DNA of interest can be conveniently introduced into body fluids or the bloodstream and then site specifically directed to the target organ or tissue in the body. Liposomes, for example, can be developed which are cell specific or organ specific. The foreign DNA carried by the liposome thus will be taken up by those specific cells. Injection of immunoliposomes that are targeted to a specific receptor on certain cells can be used as a convenient method of inserting the DNA into the cells bearing the receptor. Another carrier system that has been used is the asialoglycoprotein/polylysine conjugate system for carrying DNA to hepatocytes for in vivo gene transfer.

Transfected DNA may also be complexed with other kinds of carriers so that the DNA is carried to the recipient cell and then resides in the cytoplasm or in the nucleoplasm of the recipient cell. DNA can be coupled to carrier nuclear proteins in specifically engineered vesicle complexes and carried directly into the nucleus.

Carrier mediated gene transfer may also involve the use of lipid-based proteins which are not liposomes. For example, lipofectins and cytofectins are lipid-based positive ions that bind to negatively charged DNA, forming a complex that can ferry the DNA across a cell membrane. Fectins may also be used. Another method of carrier mediated gene transfer involves receptor-based endocytosis. In this method, a ligand (specific to a cell surface receptor) is made to form a complex with a gene of interest and then injected into the bloodstream; target cells that have the cell surface receptor will specifically bind the ligand and transport the ligand-DNA complex into the cell.

Biological gene therapy methodologies usually employ viral vectors to insert genes into cells. The term “vector” as used herein in the context of biological gene therapy means a carrier that can contain or associate with specific polynucleotide sequences and which functions to tansport the specific polynucleotide sequences into a cell. The transfected cells may be cells derived from the patient's normal tissue, the patient's diseased tissue, or may be non-patient cells. Examples of vectors include plasmids and infective microorganisms such as viruses, or non-viral vectors such as the ligand-DNA conjugates, liposomes, and lipid-DNA complexes discussed above.

Viral vector systems which may be utilized in the present invention include, but are not limited to, (a) adenovirus vectors; (b) retrovirus vectors; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. In the preferred embodiment the vector is an adenovirus.

Thus, a wide variety of gene transfer/gene therapy vectors and constructs are known in the art. These vectors are readily adapted for use in the methods of the present invention. By the appropriate manipulation using recombinant DNA/molecular biology techniques to insert an operatively linked G6PD encoding nucleic acid segment (either active or inactive) into the selected expression/delivery vector, many equivalent vectors for the practice of the present invention can be generated.

Antagonists useful in the method of the present invention are G6DP antibodies, including monoclonal, chimeric humanized, and recombinant antibodies and fragment thereof which are characterized by high affinity binding to G6DP in vivo and low toxicity. Neutralizing antibodies are readily raised in animals such as rabbits or mice by immunization with G6DP. Immunized mice are particularly useful for providing sources of B cells for the manufacture of hybridomas, which in turn are cultured to produce large quantities of anti-G6DP monoclonal antibodies. Chimeric antibodies are immunoglobin molecules characterized by two or more segments or portions derived from different animal species. Generally, the variable region of the chimeric antibody is derived from a non-human mammalian antibody, such as murine monoclonal antibody, and the immunoglobin constant region is derived from a human immunoglobin molecule. Preferably, both regions and the combination have low immunogenicity as routinely determined. Humanized antibodies are immunoglobin molecules created by genetic engineering techniques in which the murine constant regions are replaced with human counterparts while retaining the murine antigen binding regions. The resulting mouse-human chimeric antibody should have reduced immunogenicity and improved pharmacokinetics in humans. Preferred examples of high affinity monoclonal antibodies and chimeric derivatives thereof, useful in the methods of the present invention, are described in the European Patent Application EP 186,833; PCT Patent Application WO 92/16553; and U.S. Pat. No. 6,090,923.

RNA interference or “RNAi” is a term initially coined by Fire and co-workers to describe the observation that double-stranded RNA (dsRNA) can block gene expression when it is introduced into worms (Fire et al. (1998) Nature 391, 806-811). dsRNA directs gene-specific, post-transcriptional silencing in many organisms, including vertebrates, and has provided a new tool for studying gene function. RNAi involves mRNA degradation of a target gene. Results showed that RNAi is ATP-dependent yet uncoupled from mRNA translation. That is, protein synthesis is not required for RNAi in vitro. In the RNAi reaction, both strands (sense and antisense) of the dsRNA are processed to small RNA fragments or segments of from about 21 to about 23 nucleotides (nt) in length (RNAs with mobility in sequencing gels that correspond to markers that are 21-23 nt in length, optionally referred to as 21-23 nt RNA). Processing of the dsRNA to the small RNA fragments does not require the targeted mRNA, which demonstrates that the small RNA species is generated by processing of the dsRNA and not as a product of dsRNA-targeted mRNA degradation. The mRNA is cleaved only within the region of identity with the dsRNA. Cleavage occurs at sites 21-23 nucleotides apart, the same interval observed for the dsRNA itself, suggesting that the 21-23 nucleotide fragments from the dsRNA are guiding mRNA cleavage. Isolated RNA molecules (double-stranded; single-stranded) of from about 21 to about 23 nucleotides mediate RNAi. That is, the isolated RNAs mediate degradation of mRNA of a gene to which the mRNA corresponds (mediate degradation of mRNA that is the transcriptional product of the gene, which is also referred to as a target gene). Isolated RNA molecules specific to G6PD mRNA, which mediate RNAi, are antagonists useful in the method of the present invention.

It will be appreciated by those of skill that cloned genes readily can be manipulated to alter the amino acid sequence of a protein. The cloned gene for G6DP can be manipulated by a variety of well known techniques for in vitro mutagenesis, among others, to produce variants of the naturally occurring human protein, herein referred to as muteins, that may be used in accordance with the invention.

The variation in primary structure of muteins of G6DP useful in the invention, for instance, may include deletions, additions and substitutions. The substitutions may be conservative or non-conservative. The differences between the natural protein and the mutein generally conserve desired properties, mitigate or eliminate undesired properties and add desired or new properties.

Similarly, techniques for making small oligopeptides and polypeptides that exhibit activity of larger proteins from which they are derived (in primary sequence) are well known and have become routine in the art. Thus, peptide analogs of proteins of the invention, such as peptide analogs of G6DP that exhibit antagonist activity also are useful in the invention.

Mimetics also can be used in accordance with the present invention to modulate angiogenesis. The design of mimetics is known to those skilled in the art, and is generally understood to be peptides or other relatively small molecules that have an activity the same or similar to that of a larger molecule, often a protein, on which they are modeled.

Variations and modifications to the above protein, inhibitors, antibodies and vectors to increase or decrease G6PD expression can be linked with a molecular counterligand for endothelial cell adhesion molecules, such as PECAM-G6PD, to make these agents tissue specific.

In one aspect, the present invention provides for a method for the modulation of angiogenesis in a tissue associated with a disease process or condition, and thereby affect events in the tissue which depend upon angiogenesis. Generally, the method comprises administering to the tissue, associated with, or suffering from a disease process or condition, an angiogenesis-modulating amount of a composition comprising a G6PD protein or a nucleic acid vector expressing active or a G6PD antagonist.

Any of a variety of tissues, or organs comprised of organized tissues, can support angiogenesis in disease conditions including skin, muscle, gut, connective tissue, brain tissue, nerve cells, joints, bones and the like tissue in which blood vessels can invade upon angiogenic stimuli.

The patient to be treated according to the present invention in its many embodiments is a human patient, although the invention is effective with respect to all mammals. In this context, a “patient” is a human patient as well as a veterinary patient, a mammal of any mammalian species in which treatment of tissue associated with diseases involving angiogenesis is desirable, particularly agricultural and domestic mammalian species.

Thus, the method embodying the present invention comprises administering to a patient a therapeutically effective amount of a physiologically tolerable composition containing a G6PD protein or nucleic acid vector for expressing a G6PD protein or an antagonist thereof.

The dosage ranges for the administration of a G6PD protein or antagonist depend upon the form of the protein, and its potency, as described further herein, and are amounts large enough to produce the desired effect in which angiogenesis and the disease symptoms mediated by angiogenesis are ameliorated. The dosage should not be so large as to cause adverse side effects, such as hyperviscosity syndromes, pulmonary edema, congestive heart failure, and the like. Generally, the dosage will vary with the age, condition, sex and extent of the disease in the patient and can be determined by one of skill in the art. The dosage can also be adjusted by the individual physician in the event of any complication.

A therapeutically effective amount is an amount of G6PD protein, or nucleic acid encoding for (active or inactive) G6PD protein or G6PD antagonist, sufficient to produce a measurable modulation of angiogenesis in the tissue being treated, i.e., angiogenesis-modulating amount. Modulation of angiogenesis can be measured or monitored by the CAM assay, examination of tumor tissues, or by other methods known to one skilled in the art.

The G6PD protein or nucleic acid vector expressing such protein or G6PD antagonist can be administered parenterally by injection or by gradual infusion over time. Although the tissue to be treated can typically be accessed in the body by systemic administration and therefore most often treated by intravenous administration of therapeutic compositions, other tissues and delivery means are contemplated where there is a likelihood that the tissue targeted contains the target molecule. Thus, compositions of the invention can be administered intravenously, intraperitoneally, intramuscularly, subcutaneously, intracavity, transdermally, and can be delivered by peristaltic means, if desired.

The therapeutic compositions containing a G6PD protein or nucleic acid vector expressing the protein or G6PD antagonist can be conventionally administered intravenously, as by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required physiologically acceptable diluent, i.e., carrier, or vehicle.

The compositions are administered in a manner compatible with the dosage formulation, and in a therapeutically effective amount The quantity to be administered and timing depends on the subject to be treated, capacity of the subject's system to utilize the active ingredient, and degree of therapeutic effect desired.

Precise amounts of active ingredient required to be administered depend on the judgment of the practitioner and are peculiar to each individual. However, suitable dosage ranges for systemic application are disclosed herein and depend on the route of administration. Suitable regimes for administration are also variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

G6PD protein, antagonists, and vectors may be adapted for catheter-based delivery systems including coated balloons, slow-release drug-eluting stents, microencapsulated PEG liposomes, or nanobeads for delivery using direct mechanical intervention with or without adjunctive techniques such as ultrasound.

There are a variety of diseases in which inhibition of angiogenesis is important, referred to as angiogenic diseases, including but not limited to, inflammatory disorders such as immune and non-immune inflammation, chronic articular rheumatism and psoriasis, disorders associated with inappropriate or inopportune invasion of vessels such as diabetic retinopathy, neovascular glaucoma, restenosis, capillary proliferation in atherosclerotic plaques and osteoporosis, and cancer associated disorders, such as solid tumors, solid tumor metastases, angiofibromas, retrolental fibroplasia, hemangiomas and Kaposi sarcoma

Thus, methods which inhibit angiogenesis in a tissue associated with a disease condition ameliorates symptoms of the disease and, depending upon the disease, can contribute to cure of the disease. In one embodiment, the invention contemplates inhibition of angiogenesis, per se, in a tissue associated with a disease condition. The extent of angiogenesis in a tissue, and therefore the extent of inhibition achieved by the present methods, can be evaluated by a variety of methods.

Thus, in one embodiment, a tissue to be treated is an inflamed tissue and the angiogenesis to be inhibited is inflamed tissue angiogenesis where there is neovascularization of inflamed tissue. This particular method includes inhibition of angiogenesis in arthritic tissues, such as in a patient with chronic articular rheumatism, in immune or non-immune inflamed tissues, in psoriatic tissue, and the like.

In another embodiment, a tissue to be treated is a retinal tissue of a patient suffering from a retinal disease such as diabetic retinopathy or neovascular glaucoma and the angiogenesis to be inhibited is retinal tissue angiogenesis where there is neovascularization of retinal tissue.

In an additional embodiment, a tissue to be treated is a tumor tissue of a patient with a solid tumor, a metastases, a skin cancer, a breast cancer, a hemangioma or angiofibroma and the like cancer, and the angiogenesis to be inhibited is tumor tissue angiogenesis where there is neovascularization of a tumor tissue. Typical solid tumor tissues treatable by the present methods include lung, pancreas, breast, colon, laryngeal, ovarian, and the like tissues. Inhibition of tumor tissue angiogenesis is a particularly preferred embodiment because of the important role neovascularization plays in tumor growth. In the absence of neovascularization of tumor tissue, the tumor tissue does not obtain the required nutrients, slows in growth, ceases additional growth, regresses and ultimately becomes necrotic resulting in killing of the tumor.

Stated in other words, the present invention provides for a method of inhibiting tumor neovascularization by inhibiting tumor angiogenesis according to the present methods. Similarly, the invention provides a method of inhibiting tumor growth by practicing the angiogenesis-inhibiting methods.

The methods are also particularly effective against the formation of metastases because (1) their formation requires vascularization of a primary tumor so that the metastatic cancer cells can exit the primary tumor and (2) their establishment in a secondary site requires neovascularization to support growth of the metastases.

The G6PD antagonist of the invention may be combined with a therapeutically effective amount of an angiogenesis inhibiting factor, for example but not limited to, another molecule which negatively regulates angiogenesis which may be, but is not limited to, platelet factor 4, thrombospondin-1, tissue inhibitors of metalloproteases (TIMP1 and TIMP2) prolactin (16-Kd fragment), angiostatin (38-Kd fragment of plasminogen), bFGF soluble receptor, transforming growth factor-beta, interferon alfa, and placental proliferin-related protein.

In a yet further embodiment, the invention contemplates the practice of the method in conjunction with other therapies such as conventional chemotherapy directed against solid tumors and for control of establishment of metastases as well as other forms of antiangiogenesis therapy. The administration of angiogenesis inhibitor is typically conducted during or after chemotherapy, although it is preferably to inhibit angiogenesis after a regimen of chemotherapy at times where the tumor tissue will be responding to the toxic assault by inducing angiogenesis to recover by the provision of a blood supply and nutrients to the tumor tissue. In addition, it is preferred to administer the angiogenesis inhibition methods after surgery where solid tumors have been removed as a prophylaxis against metastases.

Insofar as the present methods apply to inhibition of tumor neovascularization, the methods can also apply to inhibition of tumor tissue growth, to inhibition of tumor metastases formation, and to regression of established tumors.

Restenosis is a process of smooth muscle cell (SMC) migration and proliferation into the tissue at the site of percutaneous transluminal coronary angioplasty which hampers the success of angioplasty. The migration and proliferation of SMC's during restenosis can be considered a process of angiogenesis which is inhibited by the present methods. Therefore, the invention also contemplates inhibition of restenosis by inhibiting angiogenesis according to the present methods in a patient following angioplasty procedures. For inhibition of restenosis, the inactivated G6PD is typically administered after the angioplasty procedure because the coronary vessel wall is at risk of restenosis, typically for from about 2 to about 28 days, and more typically for about the first 14 days following the procedure.

The present method for inhibiting angiogenesis in a tissue associated with a disease condition, and therefore for also practicing the methods for treatment of angiogenesis-related diseases, comprises contacting a tissue in which angiogenesis is occurring, or is at risk for occurring, with a therapeutically effective amount of a composition comprising an G6PD antagonist.

In cases where it is desirable to promote or potentiate angiogenesis, administration of an active G6PD protein to the tissue is useful. The routes and timing of administration are comparable to the methods described above for inhibition. The G6PD protein may be used in conjunction with other angiogenesis promoting agents, e.g., VEGF or bFGF.

The present invention provides therapeutic compositions useful for practicing the therapeutic methods described herein. Therapeutic compositions of the present invention contain a physiologically tolerable carrier together with a G6DP protein or vector capable of expressing a G6DP protein or G6PD antagonist as described herein, dissolved or dispersed therein as an active ingredient. In a preferred embodiment, the therapeutic composition is not immunogenic when administered to a mammal or human patient for therapeutic purposes.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like.

The preparation of a pharmacological composition that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions can also contain liquid phases in addition to and to the exclusion of water. Exemplary of such additional liquid phases are glycerin, vegetable oils such as cottonseed oil, and water-oil emulsions.

For topical application, the G6PD antagonist may be combined with a carrier so that an effective dosage is delivered, based on the desired activity ie., ranging from an effective dosage, for example, about 1.0 μM to 1.0 mM to prevent limitation, an ointment, cream, gel, paste, foam, aerosol, suppository, pad or gelled stick.

The amount of the active G6PD protein or G6PD antagonist (referred to as “agents”) used in the invention that will be effective in the treatment of a particular disorder or condition will depend on the nature of the disorder or condition, and can be determined by standard clinical techniques. In addition, in vitro assays such as those discussed herein may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for administration of agents are generally about 0.01 pg/kg body weight to 1 mg/kg body weight. Effective doses may be extrapolated from dose-response curves derived from in vitro or animal model test bioassays or systems.

Administration of the doses recited above can be repeated. In a preferred embodiment, the doses recited above are administered 2 to 7 times per week. The duration of treatment depends upon the patient's clinical progress and responsiveness to therapy.

The invention also contemplates an article of manufacture which is a labeled container for providing a G6DP protein or antagonist of the invention. An article of manufacture comprises packaging material and a pharmaceutical agent contained within the packaging material.

The pharmaceutical agent in an article of manufacture is any of the compositions of the present invention suitable for providing a G6DP protein or antagonist and formulated into a pharmaceutically acceptable form as described herein according to the disclosed indications. Thus, the composition can comprise a G6DP protein or a DNA molecule which is capable of expressing the protein or an antagonist.

The article of manufacture contains an amount of pharmaceutical agent sufficient for use in treating a condition indicated herein, either in unit or multiple dosages.

The packaging material comprises a label which indicates the use of the pharmaceutical agent contained therein, e.g., for treating conditions assisted by the inhibition or potentiation of angiogenesis, and the like conditions disclosed herein.

The label can further include instructions for use and related information as may be required for marketing. The packaging material can include container(s) for storage of the pharmaceutical agent

As used herein, the term packaging material refers to a material such as glass, plastic, paper, foil, and the like capable of holding within fixed means a pharmaceutical agent Thus, for example, the packaging material can be plastic or glass vials, laminated envelopes and the like containers used to contain a pharmaceutical composition including the pharmaceutical agent.

In preferred embodiments, the packaging material includes a label that is a tangible expression describing the contents of the article of manufacture and the use of the pharmaceutical agent contained therein.

The references cited throughout this application are herein incorporated by reference.

It is understood that the foregoing detailed description and the following examples are illustrative only and are not to be taken as limitations upon the scope of the invention. Various changes and modifications to the disclosed embodiments, which will be apparent to those skilled in the art, may be made without departing from the spirit and scope of the present invention. Further, all patents, patent applications and publications cited herein are incorporated herein by reference.

### EXAMPLE 1

Methodology to increase G6PD expression and activity include direct injection of G6PD DNA (“naked DNA”) and adenoviral-mediated gene transfer. We will use an adenoviral vector incorporating the cDNA for rat G6PD or cDNA for human G6PD, to increase G6PD expression. To construct this vector, rat or human G6PD cDNA is excised from its plasmid and inserted into a shuttle vector, pHIHG-Ad2. The resulting plasmid is then digested with Pac I and Mfe I and transferred into an E. coli BJ5183 strain together with a CIa I linearized adenoviral vector, pAd-hGM-CSF. This vector contains the entire adenoviral genomic sequence except the E1 and E3 regions. Homologous recombination in the E. coli strain produced a recombinant adenoviral vector, pAd-G6PD, which was extracted and transferred to E. coli DH5α for large-scale plasmid production. The sequence of the vector is confirmed by restriction digestion. PAD-G6PD is linearized with Pac I and transfected into 293 cells using Lipofectamine® (Gibco). G6PD activity is measured and expression confirmed by Western analysis. For full-scale preparation, following a 4-hr incubation at 37° C. with pAd-G6PD, transfected 293 cells are placed in full growth media and harvested when the cells become rounded due to viral cytopathic effect. The cell pellet is then resuspended in glycerol 10% and recombinant virus will be extracted by repeated freeze-thaw.

### EXAMPLE 2

G6PD and Endothelial Cell Proliferation

To investigate the role of G6PD in vascular endothelial cell proliferation, we initially sought to determine if growth factors involved in angiogenesis, such as vascular endothelial growth factor (VEGF), influenced G6PD activity. To examine this effect, we treated bovine aortic endothelial cells (BAEC) with vascular endothelial growth factor (VEGF)(100 ng/ml), an essential growth factor for angiogenesis, and, compared to untreated cells, demonstrated a significant increase in G6PD activity after 4 hr (60.9±18 vs. 182.7±37 units/6 min/mg protein, p<0.01, n=6), that returned toward baseline by 24 hr (92.0±22 vs. 87±19 units/6 min/mg protein, p=NS, n-6). Increased BAEC G6PD activity following stimulation with VEGF was paralleled by an increase in cellular NADPH levels at 4 hr (0.3±0.02 vs. 0.5±0.04 mmoles/mg protein, p<0.01, n—3), which similarly returned toward baseline at 24 hr (0.3±0.08 vs. 0.4±0.08 mmoles/mg protein, p=NS, n=3). Western analysis revealed that VEGF did not increase G6PD expression at either time point (data not shown). These observations suggested that VEGF increased basal G6PD activity to enhance NADPH levels, and did not stimulate de novo synthesis of G6PD.

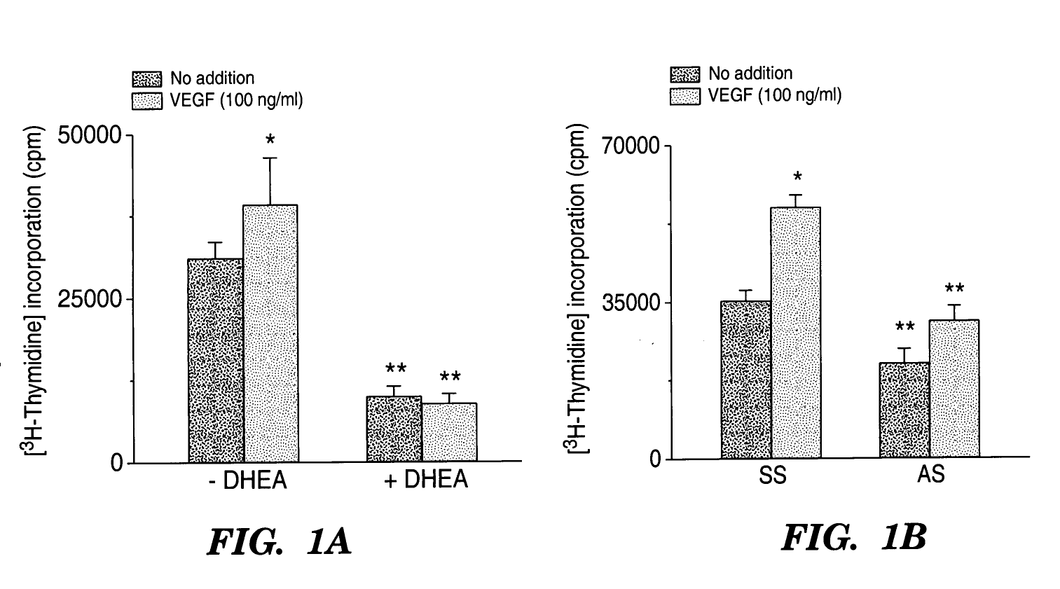

To examine further the role of G6PD in VEGF-mediated cell proliferation, we next inhibited G6PD activity by exposing BAEC to dehydroepiandrosterone (DHEA) (100 pmol/L), a noncompetitive inhibitor of G6PD, for 24 hr. We have previously demonstrated that DHEA-treated BAEC have a 60% reduction in G6PD activity resulting in a 30% decrease in the level of cellular NADPH. Compared to control cells, DHEA-treated BAEC demonstrated a significant decrease in [³H]-thymidine incorporation under basal conditions (31,125±2,410 vs. 9,967±1,531 cpm, p<0.01, n=4), and following stimulation with VEGF (100 ng/ml for 8 hr) (39,223±7,158 vs. 8,902±1,451 cpm, p<0.01, n=4) (FIG. 1A). These results were not the result of increased cell death as determined by lactate dehydrogenase activity measured in the media

To confirm these results, G6PD expression was inhibited in BAEC by transfection with an antisense phosphorothioate oligodeoxynucleotide to G6PD mRNA (5′-AGGUCACCCGAUGCACCCAUGAUGA-3′ (SEQ ID NO: 1)) (AS) using Oligofectin I (Sequitor, Inc.) as a vehicle. As a control, we used a phosphorothioate oligodeoxynucleotide with a scrambled sequence (SS). We have previously demonstrated that following transfection for 5 hours, Western analysis revealed a significant decrease in G6PD expression that was associated with a 50% reduction in G6PD activity and a 40% decrease in NADPH levels. Compared to SS-transfected BAEC, AS-transfected BAEC, with decreased G6PD expression, demonstrated decreased basal (35,258±2,368 vs. 21,109±3,284 cpm, p<0.01, n=3) and VEGF-stimulated (56,038±2,775 vs. 30,667±3,361 cpm, p<0.01, n=3):cell proliferation (FIG. 1B).

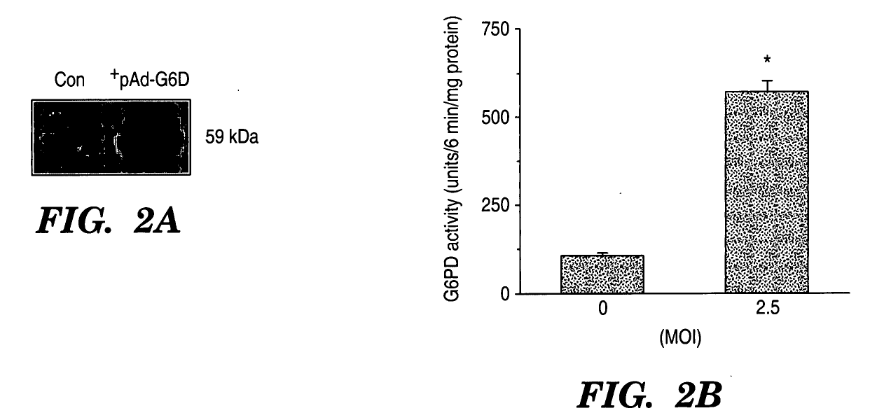

We have recently constructed an adenoviral vector incorporating murine G6PD cDNA (93% homologous to the rat cDNA). The sequence of this vector, pAd-G6PD, was confirmed by restriction digestion, and expression of the active enzyme was demonstrated in E. coli lysates. We have successfully trans(in)fected BAEC and, compared to cells trans(in)fected with empty vector, shown a significant increase in G6PD expression, activity (108.3±6.7 vs. 571.2±30.8 units/6 min/mg protein, p<0.01, n=6) (FIG. 2), and cellular NADPH levels (0.5±0.01 vs. 0.67±0.02 mmoles/mg protein, p<0.01, n=3). Gene transfer of G6PD resulted in a significant increase in cell proliferation as determined by [³H]-thymidine incorporation (31,983±3,009 vs. 51,769±2,784 cpm, p<0.01, n=3).

G6PD and Tube Formation

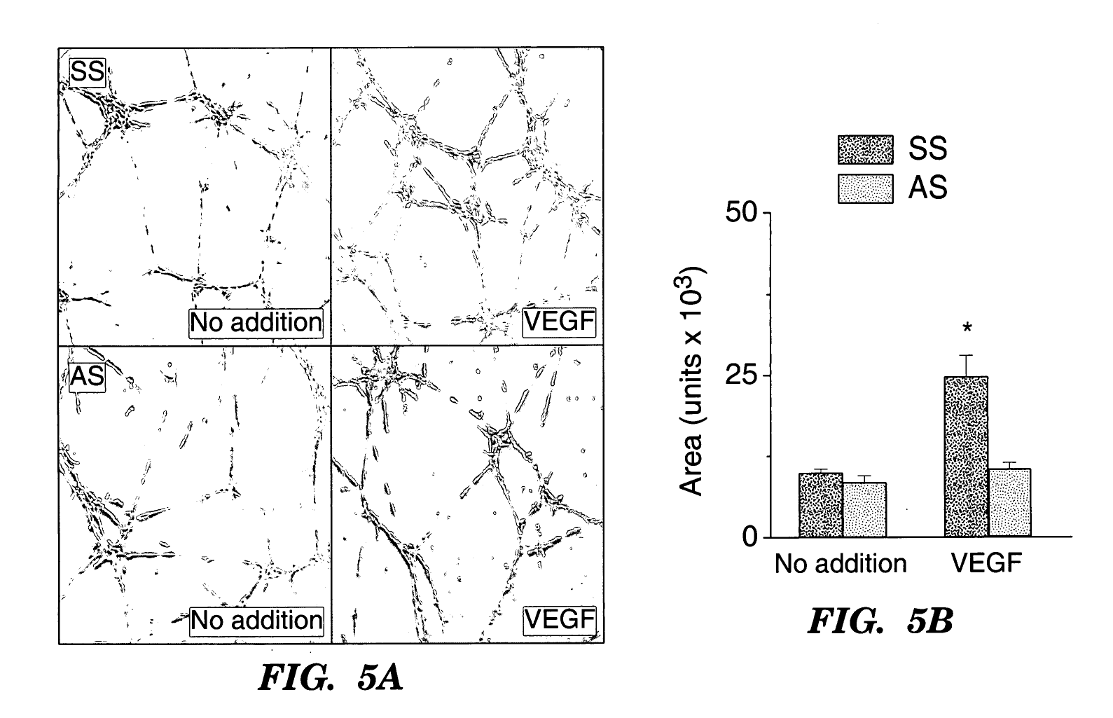

To examine the role of G6PD in endothelial cell tube formation in vitro, we plated BAEC on Matrigel and stimulated cells with VEGF (100ng/ml) for 12 hr. Results were quantified using NIH Image to determine area of tube formation. Under basal conditions, BAEC plated on Matrigel formed tubes and networks that were increased following exposure to VEGF. VEGF-mediated tube formation was significantly decreased in As-transfected BAEC compared to cells transfected with a scrambled control, and this correlated with a significant decrease in vessel area (FIG. 5). Similar results were obtained for DHEA-treated BAEC, demonstrating that G6PD importantly modulates endothelial cell tube formation.

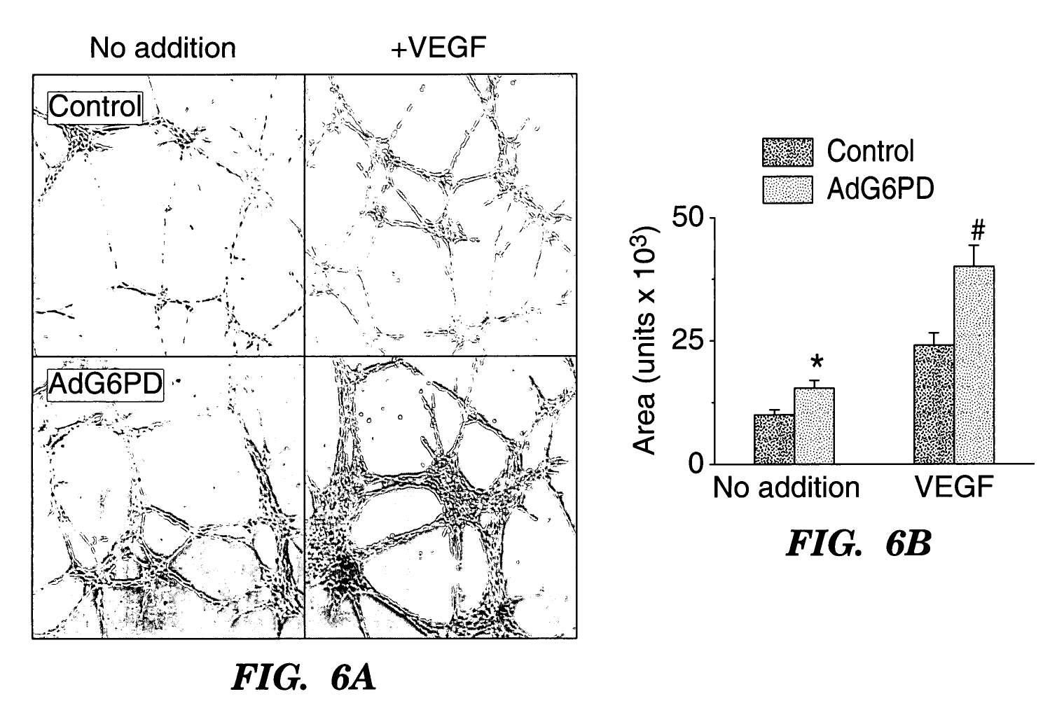

In order to confirm that G6PD importantly modulates tube formation in vitro, we next increased G6PD expression via adenoviral-mediated gene transfer and examined tube formation. Increased G6PD expression significantly increased endothelial cell tube under basal conditions as well as following VEGF stimulation (FIG. 6).

G6PD and Reactive Oxygen Species

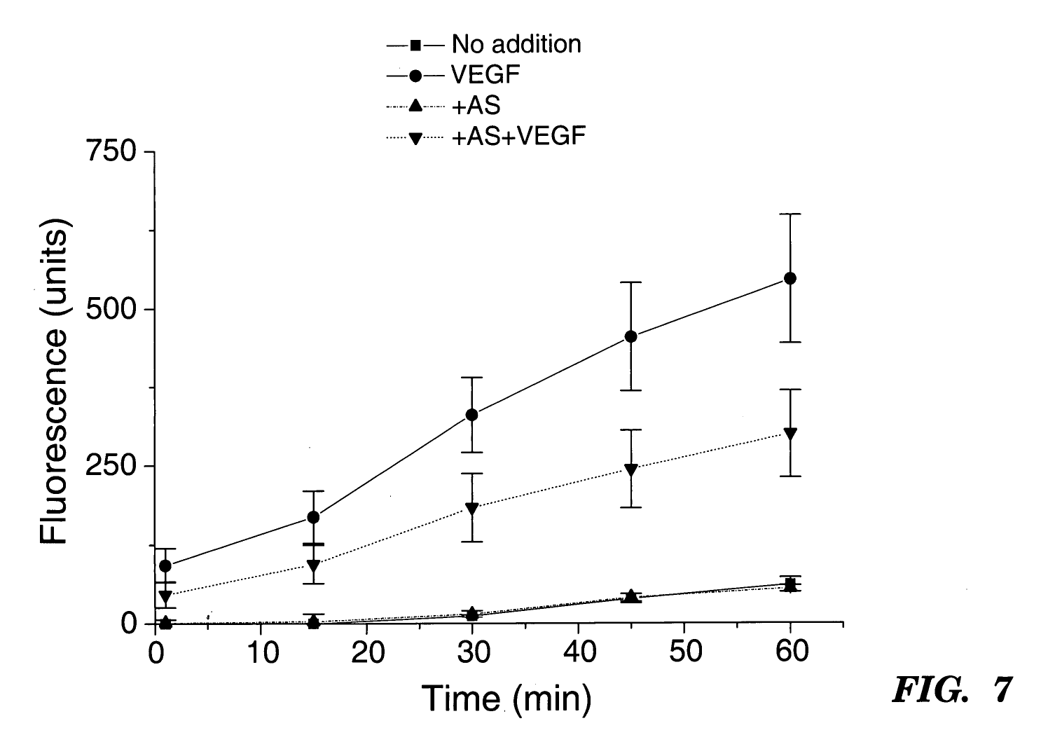

As growth factors have been suggested to increase cell proliferation and migration via the generation of reactive oxygen species (ROS), we next examined the effect of VEGF on ROS production in BAEC using 2′,7′-dichlorofluorescein diacetate fluorescence. BAEC were exposed to VEGF (100 ng/ml) for 4 hr, after which ROS formation was measured every 15 min over the course of 1 hr. At 1 hr., BAEC stimulated with VEGF demonstrated an increase in ROS formation compared to untreated cells (61.8±101.5 units, p<0.01, n=8), and, interestingly, this response was abrogated in AS-transfected BAEC (299±68.9 units, p<0.05, n=8) (FIG. 7). These observations suggested that ROS formation may play an important role in VEGF-mediated cell proliferation and deficient G6PD activity influences this response.

G6PD and Endothelial Cell Migration

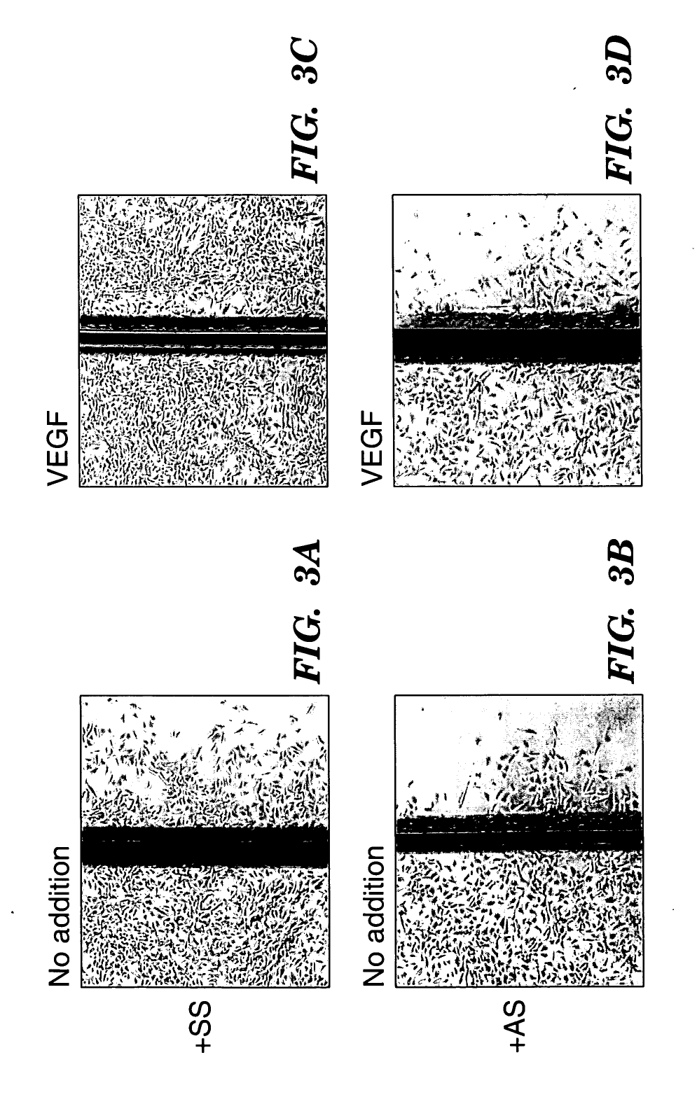

We next examined the role of G6PD on VEGF-stimulated endothelial cell migration. In a cell-wounding migration assay, BAEC were grown to confluence on a P100 culture dish. Using a sterile scalpel, a vertical incision was made in the midline of the dish, and under microscopic guidance, BAEC were removed from one-half of the plate. BAEC were then incubated with VEGF (100 ng/ml) in serum-free media, and cell migration across the midline was observed after 18 hr and quantified by the average cell count in 5 high powered fields. VEGF significantly increased BAEC migration (14±4 vs. 28±6 cells/hpf, p<0.01, n=5), an effect that was significantly decreased in BAEC pretreated with DHEA compared to untreated cells (38.6±6 vs. 10±2 cells/hpf, p<0.01, n=5) or transfected with AS compared to SS-transfected cells (52±11 vs. 16±5 cells/hpf, p<0.01, n=5). A representative cell-wounding migration assay in BAEC transfected with SS or AS is shown in FIG. 3.

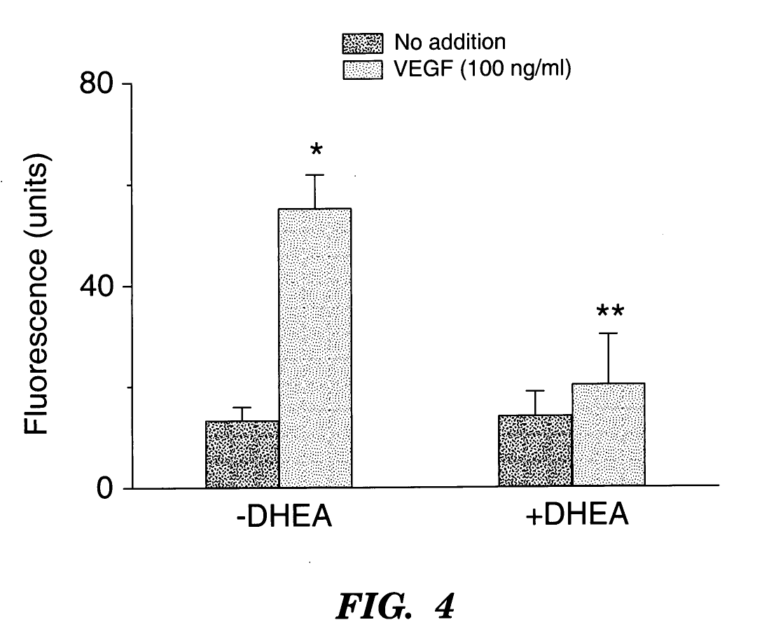

To confirm these observations, we analyzed cell migration using a modified Boyden chamber. BAEC were fluorescently labeled with calcein AM (Molecular Probes, Inc.) and 0.4×10³ cells were loaded to the top of a pre-fitted membrane filter that fit a specially designed 96-well microplate. The microplate wells were loaded with serum-free media and VEGF (100 ng/ml) for 18 hr., and cells were allowed to migrate across the membrane. The plate was then analyzed using a fluorescent microplate reader. In BAEC treated with VEGF, there was a significant increase in fluorescence, indicating an increase in cell migration, that was abrogated in DHEA-treated BAEC (FIG. 4).

G6PD and Nitric Oxide

Nitric oxide has been implicated as a second messenger important for endothelial cell proliferation and migration. We have previously established that G6PD modulates NO levels, and that decreased G6PD activity or expression is associated with a reduction in bioavailable NO. To determine if increased G6PD activity would restore bioavailable NO levels, we transfected BAEC with pAdG6PD and examined markers of NO production. Nitric oxide bioactivity was increased in AdG6PD BAEC as demonstrated by cGMP production (1.8±0.1 vs. 2.7±0.3 pmol cGMP/mg protein, p<0.04) and nitrate production (865.91±93.3 vs. 1232.2±221.2 pmol nitrate/mg protein, p<0.001) in response to bradykinin. These observations suggested that G6PD and eNOS activity function in parallel, and owing to the absolute requirement of NADPH by eNOS, we hypothesized that G6PD and eNOS may colocalize.

To examine this hypothesis, BAEC were grown to confluence on slides and stimulated with A23187 (5 μmol/L) for 10 min. Cells were then exposed to anti-G6PD and anti-eNOS antibodies, and blocked with fluorescein and rhodamine secondary antibodies. Cells were visualized using confocal microscopy.

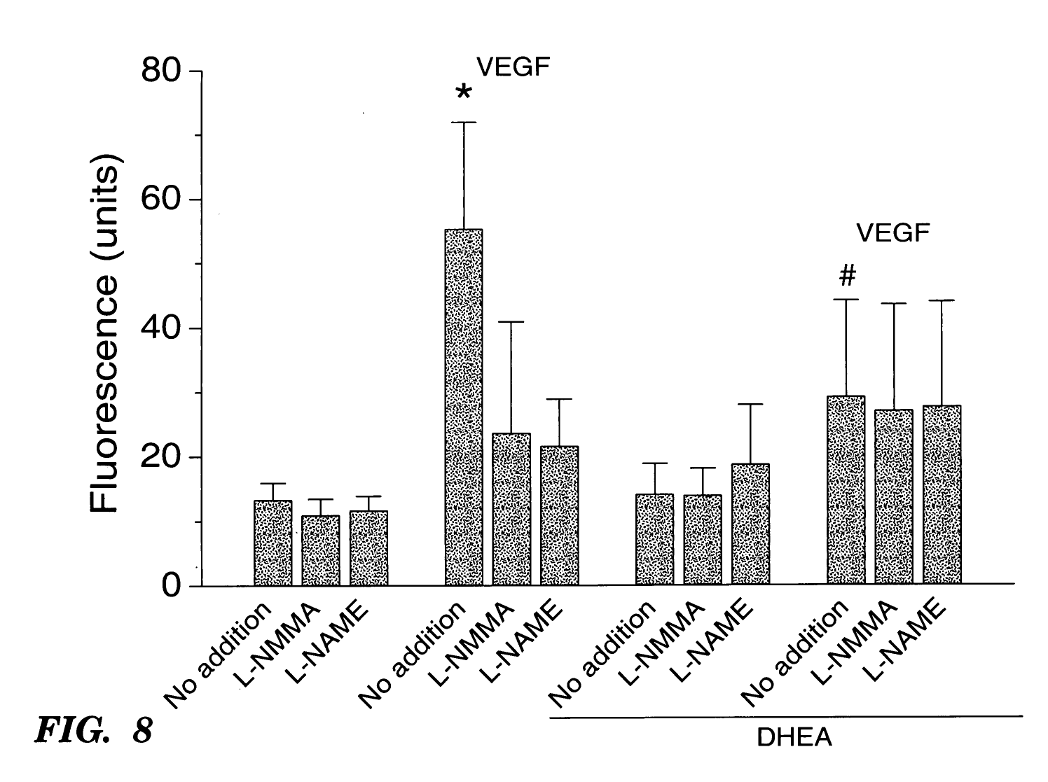

To examine the functional significance of G6PD and NO with respect to cell migration, DHEA-treated BAEC were stimulated with VEGF (100/ng/ml) for 18 her., in the presence of absence of eNOS inhibitors, L-Name (1 mmol/L), which inhibits both NO and ROS formation by eNOS, and —NMMA (100 μmol/L), which only inhibits NO formation. Migration was assayed using a modified Boyden chamber as described above (FIG. 8). Inhibition of eNOS with L-NAME (55.3±16.6 vs. 23.6±17 units, p<0.05, n=6) or L-NMMA (55.3±16.6 vs. 21.6±7.3 units, p<0.04, n-6) significantly decreased cell migration in VEGF-stimulated cells compared to control cells. DHEA-treated BAEC, VEGF did not significantly increase migration.

### EXAMPLE 3

G6PD-Deficient Murine Model

All efforts to create a G6PD knockout mouse have been unsuccessful owing to the embryonic lethality of deletion of this gene; however, the Pretsch mouse, a murine model of G6PD deficiency, demonstrates significantly reduced G6PD expression and activity. G6PD is an X-linked gene, and therefore, females may be either homozygous or heterozygous for the mutation while male offspring are hemizygous. This mutation is stably transmitted to offspring, and, through direct sequencing of PCR-amplified genomic DNA, and comparison with genomic DNA from other mouse strains, a single base difference (A-T to T-A) was identified in exon 1. We have developed G6PD mouse primers and are able to successfully genotype our animals. In addition, we have correlated the G6PD deficient genotype with a G6PD deficient phenotype in hepatic lysates. Compared to C3H wild-type mice with 100% G6PD activity, homozygous female mice have 31% activity, heterozygous female mice have 52% activity, and hemizygous male mice have 23% activity. We measured NADPH levels in hemizygous male mice and found a 50% decrease in NADPH levels in compared to wild-type mice. These observations confirm that the Pretsch mouse is a reliable model of G6PD deficiency that may be easily genotyped and is phenotypically characterized by reduced G6PD activity and decreased NADPH levels.

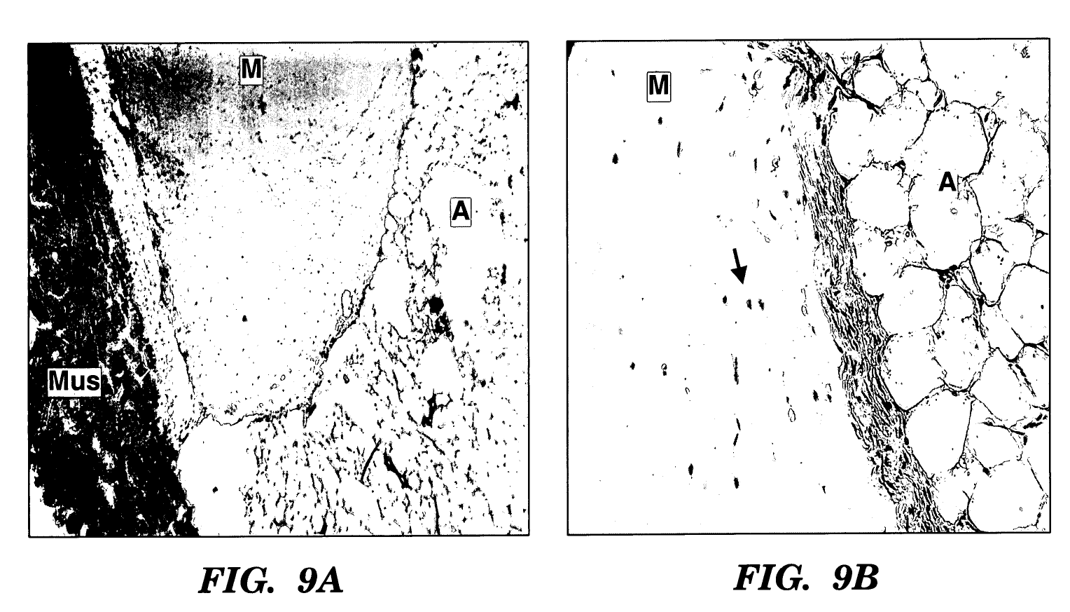

To examine the effect of G6PD activity on angiogenesis in vivo, we performed an in vivo Matrigel migration assay in C3H wild-type (WT) and hemizygous male (HEMI) mice. Matrigel, derived from mouse basement membrane proteins, was implanted subcutaneously in the groin area to form a Matrigel “plug”. Each mouse was injected on one side with either Matrigel alone, or Matrigel supplemented with VEGF (100 ng/ml). In this manner, each animal could serve as its own control. Plugs were left in place for 14 days. Following this time, the animals were euthanized, plugs excised with surrounding tissue for orientation, and embedded and sectioned in paraffin. Sections were then stained with Hematoxylin & Eosin (H&E). Images were captured digitally and assessed for cell counts. For orientation purposes, a representative Matrigel plug from a WT mouse at 4× (right) and 20× (left) magnification is shown below (FIG. 9).

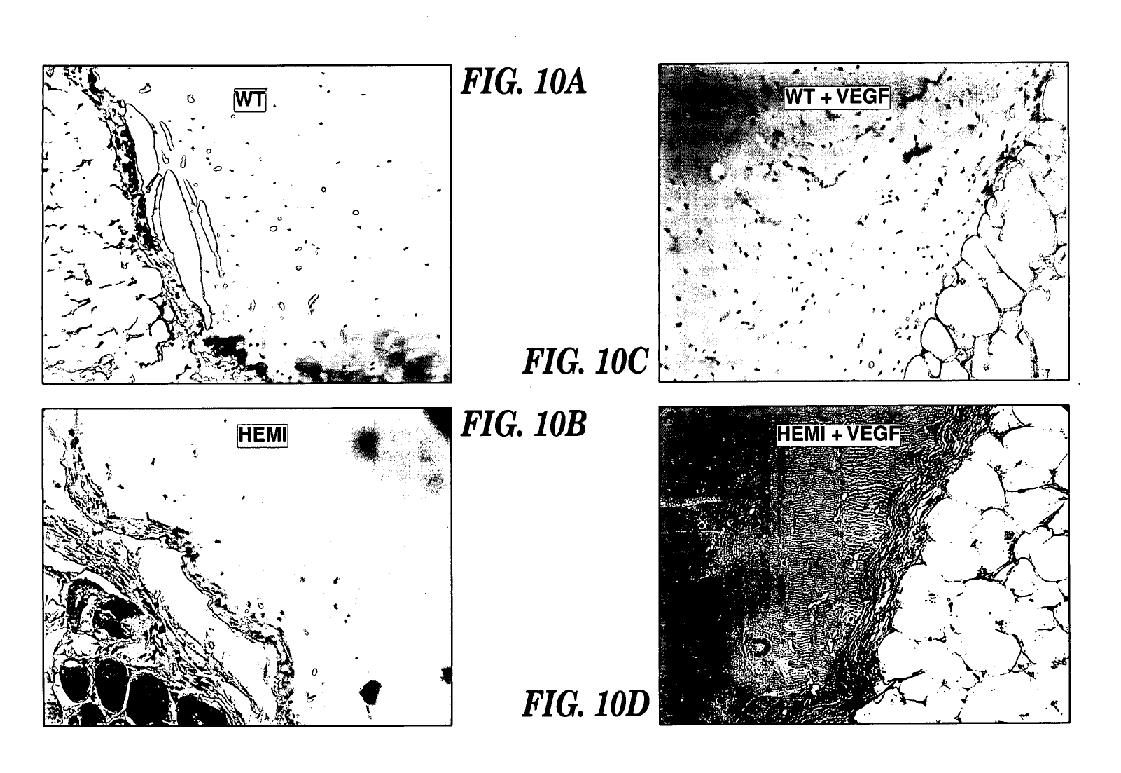

We next implanted Matrigel plugs for 14 days in WT and HEMI mice to determine the effect of G6PD on angiogenesis in vivo (FIG. 10). In WT mice, there is noticeable cell migration into the Matrigel plug (upper left), and this response is increased in Matrigel supplemented with VEGF (upper right). In contrast, in HEMI mice, there is a marked decrease in cell migration into the Matrigel plug compared to WT mice (bottom left), and exposure to VEGF did not significantly improve this response.

These findings were quantified by counting cells in 10 randomly selected high-power (20×) fields per image in 3 successive images. In WT mice, VEGF increased the number of cells migrating into the Matrigel plug; however, in HEMI mice, there was a marked reduction of cell migration into the Matrigel alone, or supplemented with VEGF. These findings confirm that G6PD modulates angiogenesis in vivo.

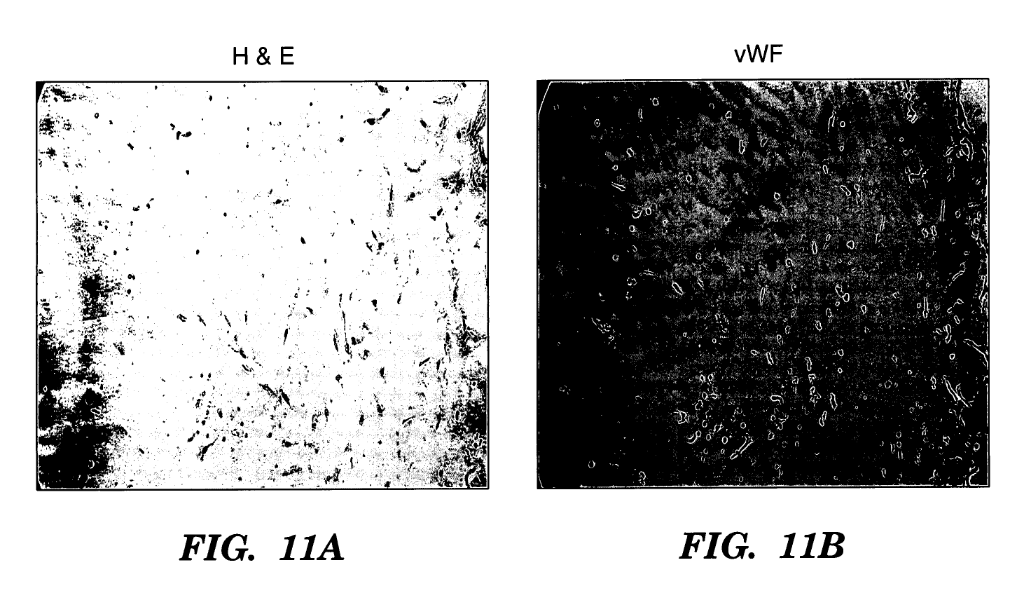

We next wanted to determine that the cells that migrated into the Matrigel were endothelial cells. Therefore, we used immunohistochemistry techniques with antigen retrieval to stain the Matrigel sections for the endothelial cell markers CD31 and von Willebrand factor (vWF) (FIG. 11). Serial sections of a Matrigel plug obtained from a WT mouse were stained with H&E (left), or for vWF antigen (right) and visualized at 20×. These findings demonstrate that the cells migrating into the Matrigel plugs are endothelial cells. Similar findings were obtained when cells were stained for CD31.

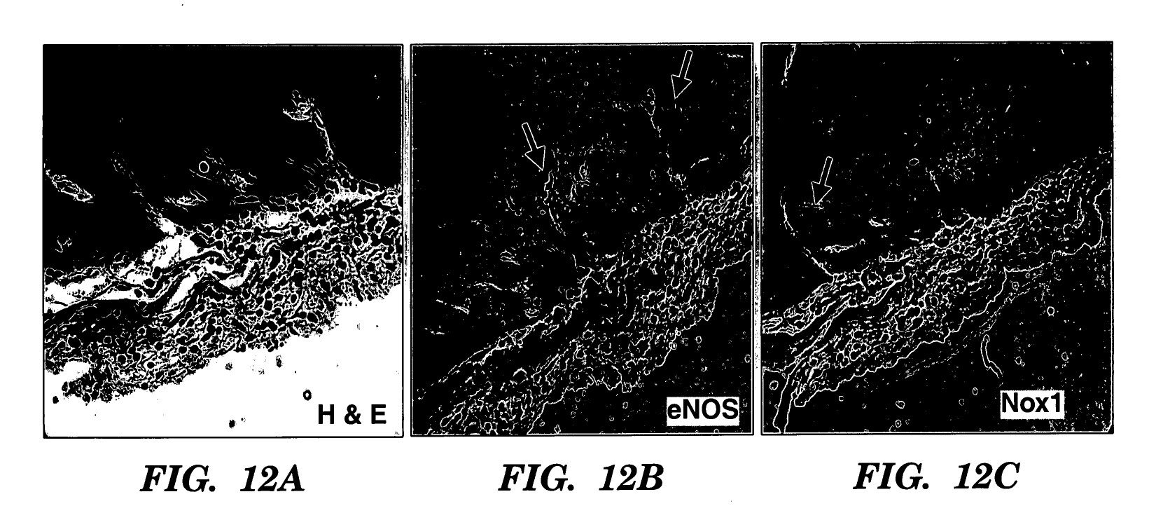

We performed further immunohistochemical analysis with antigen retrieval of serial sections of a Matrigel plug obtained from a WT mouse to confirm the presence of eNOS antigen as well as Nox1 (homologous to the catalytic subunit gp91phox of NADPH oxidase) antigen (FIG. 12). In this study, cells were initially stained for CD31 and vWF antigen to demonstrate that they were endothelial cells. These cells additionally stained positive for eNOS and Nox1 antigens as shown below.

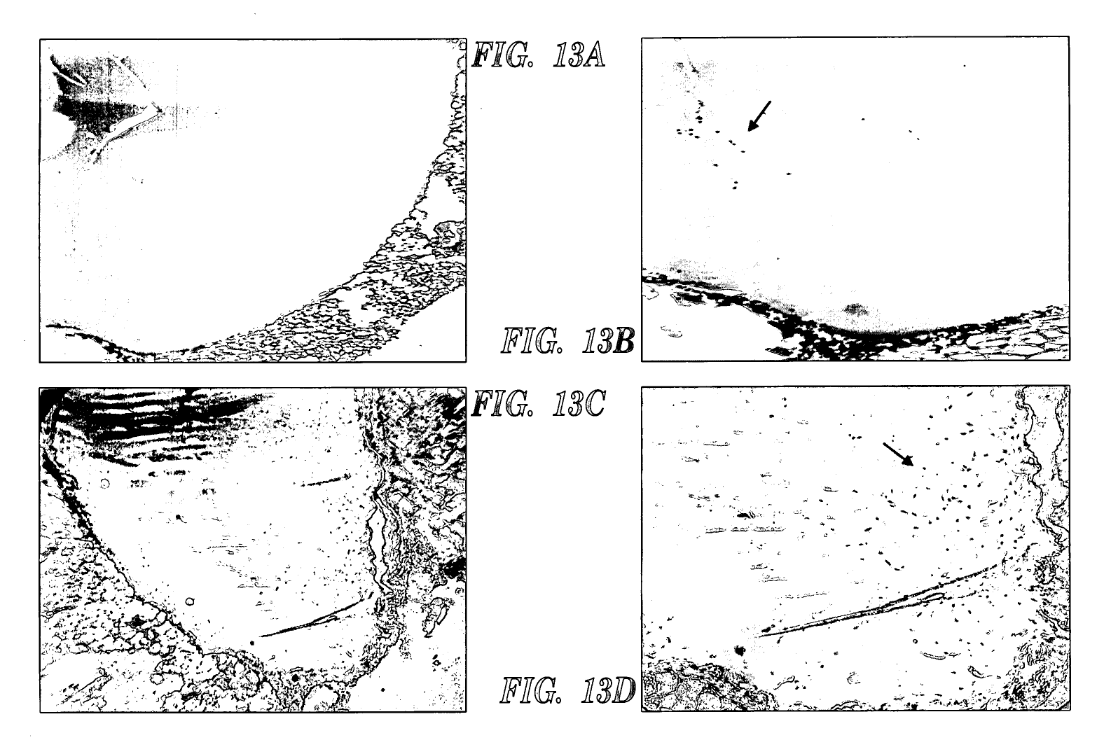

We next wanted to determine if we could “rescue” the G6PD-deficient phenotype using gene transfer to increase local G6PD expression. We treated Matrigel with an adenoviral vector encoding for G6PD implanted plugs in HEMI mice for 7 days in the presence or absence of VEGF (FIG. 13). In HEMI mice, there was minimal cell migration into the Matrigel plug (Panel A) that was not increased significantly by VEGF stimulation (Panel B). In contrast, local gene transfer of G6PD in HEMI mice resulted in a marked increase in migrating cells into the Matrigel plug (Panel C) and this effect was enhanced further by VEGF (Panel D).

Our studies demonstrate that in a murine model of G6PD deficiency we have established an in vivo measure of angiogenesis, the Matrigel plug assay and conformed that G6PD regulates angiogenesis in this model.

It will be apparent to those skilled in the art that various modifications and variations can be made to the present invention without departing from the spirit and scope of the invention. Thus, it is intended that the present invention cover the modifications and variations of this invention provided they come within the scope of the appended claims and their equivalents.

### REFERENCES

The following references and all others cited in the specification are incorporated by reference.
1. Folkman and Shing, J. Biol. Chem. 267 (16), 10931-10934 (1992).
2. Folkman, N. Engl. J Med., 285:1182-1186 (1971).
3. Yanagisawa-Miwa, et al., Science, 257:1401-1403 (1992).
4. Baffour, et al., J Vasc Surg, 16:181-91 (1992).
5. Takeshita, et al., Circulation, 90:228-234 (1994).
6. Takeshita, et al., J Clin Invest, 93:662-70 (1994).
7. O'Reilly M S, Holngren L, Shing Y, Chen C, Rosenthal R A, Moses M, Lane WS, Cao Y, Sage E H, Folkman J Angiostatin: a novel angiogenesis inhibitor that mediates the suppression of metastases by a Lewis lung carcinoma (1994) Cell 79 315-28
8. O'Reilly, M. S., Rosenthal, R., Sage, E. H., Smith, S., Holmgren, L., Moses, M., Shing, Y., and Folkman, J. The suppression of tumor metastases by a primary tumor. (1993) Surg. Forum 44 474-476
9. Ray R (1996) Molecular recognition in vitamin D-binding protein. Proc Soc Exp Biol Med 212 305-12.
10. Battegay, J. Mol. Med, 73, 333-346 (1995).
11. Hanahan et al., Cell, 86, 353-364 (1996).
12. Folkman, N. Engl. J. Med, 333, 1757-1763 (1995).

1. A method for stimulating angiogenesis in a tissue associated with a disease condition comprising administering to said tissue an angiogenesis stimulating amount of a pharmaceutical composition comprising a G6PD protein or a nucleotide sequence encoding for said protein.

2. The method of claim 1 wherein said tissue has abnormal circulation.

3. A method for inhibiting angiogenesis in a tissue associated with a disease condition comprising administering to said tissue an angiogenesis inhibiting amount of a pharmaceutical composition comprising a G6PD antagonist.

4. The method of claim 3 wherein said tissue is a solid tumor or solid tumor metastasis.

5. The method of claim 3, wherein said administering is conducted in conjunction with chemotherapy.

6. The method of claim 3, wherein said tissue is retinal tissue and said condition is retinopathy, diabetic retinopathy or macular degeneration.

7. The method of claim 3, wherein said tissue is at the site of coronary angioplasty and said tissue is at risk for restenosis.

8. The method of claim 1 or 3, wherein said administering comprises intravenous, transdermal, intrasynovial, intramuscular, or oral administration.

9. The method of claim 3, wherein said tissue is inflamed and said condition is arthritis or rheumatoid arthritis.

10. A pharmaceutical composition for stimulating angiogenesis in a target mammalian tissue comprising a gene transfer vector containing a nucleic acid, said nucleic acid having a nucleic acid segment encoding for a G6PD protein and a pharmaceutically acceptable carrier or excipient.

11. A pharmaceutical composition for inhibiting angiogenesis in a target mammalian tissue comprising a gene transfer vector containing a nucleic acid, said nucleic acid having a nucleic acid segment encoding a antisense G6PD oligonucleotide and a pharmaceutically acceptable carrier or excipient.

12. A pharmaceutical composition for stimulating angiogenesis in a target mammalian tissue comprising a therapeutic amount of a G6PD protein, and a pharmaceutically acceptable carrier or excipient.

13. A pharmaceutical composition for inhibiting angiogenesis in a target mammalian tissue comprising a therapeutic amount of a G6PD antagonist, and a pharmaceutically acceptable carrier or excipient.

14. An article of manufacture comprising packaging material and a pharmaceutical composition contained within said packaging material, wherein said pharmaceutical composition is capable of inhibiting angiogenesis in a tissue associated with a disease condition, wherein said packaging material comprises a label which indicates that said pharmaceutical composition can be used for treating disease conditions by inhibiting angiogenesis, and wherein said pharmaceutical composition comprises a G6PD antagonist.

15. An article of manufacture comprising packaging material and a pharmaceutical composition contained within said packaging material, wherein said pharmaceutical composition is capable of stimulating angiogenesis in a tissue associated with a disease condition, wherein said packaging material comprises a label which indicates that said pharmaceutical composition can be used for treating disease conditions by stimulating angiogenesis, and wherein said pharmaceutical composition comprises a G6PD protein or a vector containing a DNA segment encoding said protein.
